(19) United States
(12) Patent Application Publication
(10) Pub. No.: US 2006/0051400 A1
(43) Pub. Date: Mar. 9, 2006

(54) Steam stable enzyme compositions

(76) Inventor: Percy A. Jaquess, Cordova, TN (US)

Correspondence Address: KILYK & BOWERSOX, P.L.L.C., 400 HOLIDAY COURT, SUITE 102, WARRENTON, VA, 20186, US

(21) Appl. No.: 10/937,618

(22) Filed: Sep. 9, 2004

(51) Int. Cl. A23K 1/165; C12N 11/04; C14C 1/00

(52) U.S. Cl. 424/442; 435/182; 435/265

(57) ABSTRACT

Enzyme compositions that are steam stable and methods for producing the same are described. In one embodiment, the enzyme composition breaks down in the presence of water, thereby releasing the enzyme for catalytic function.

### BACKGROUND OF THE INVENTION

The present invention relates to enzyme formulations and methods of preparing the same. More particularly, in the present invention there is provided a protected enzyme, and a method of producing the same.

Enzymes are central to every biochemical process. They catalyze hundreds of reactions by which nutrient molecules are degraded, chemical energy is conserved and transformed, and biological macromolecules are made from simple precursors. With very few exceptions, enzymes are proteins and their catalytic activity depends on the integrity of their native protein conformation. If an enzyme is denatured or broken down into its subunits, its catalytic activity is lost. Enzymes can be denatured in a number of ways, for example, by chemical reactions, changes in pH, or through temperature variation.

The application of enzymes to foodstuffs and in manufacturing assists in digestion/nutrient absorption and to increasing the rate/effectiveness of industrial processes. Unfortunately, many processes require the application of conditions which denature enzymes. For example, the United States Food and Drug Administration requires the sterilization (often accomplished through steam treatment) of many food products before they can be brought to market. Steam treatment, while effective in destroying food borne pathogens also denatures many useful enzymes.

U.S. Pat. No. 6,342,381, incorporated herein by reference, relates to enzyme stabilization using polyamide oligomers. The compositions described in the '381 patent are generally capable of protecting enzymes up to a temperature of 50° C. but are not generally capable of protecting enzymes from higher temperatures such as those involved with steam treatment. The compositions described in the '381 patent are liquids at room temperature and the reactions described therein, are stopped before super polyamide tactile fibers are formed.

There is, therefore, a need for a protected enzyme, and a method of producing the same, which prevents an enzyme from becoming denatured at high temperatures, is non-toxic, is a solid at room temperature and also allows enzymes to function catalytically when needed.

### SUMMARY OF THE PRESENT INVENTION

It is, therefore, a feature of the present invention to provide an enzyme that is protected from denaturation.

Another feature of the present invention is to provide an enzyme that is protected from denaturation, but can be released from its protectorant.

A further feature of the present invention is to provide a protected enzyme that has a non-toxic protective element(s) and retains its catalytic function after release from the protective element.

Additional features and advantages of the present invention will be set forth in part in the description that follows, and in part will be apparent from the description, or may be learned by practice of the present invention. The objectives and other advantages of the present invention will be realized and attained by means of the elements and combinations particularly pointed out in the description and appended claims.

To achieve these and other advantages, and in accordance with the purposes of the present invention, as embodied and broadly described herein, the present invention relates to a solid heat stable enzyme composition, which includes at least one enzyme and at least one polymer. The at least one enzyme is stable even when the composition is exposed to a temperature of 100° C. or higher. The present invention further relates to food, medicine, and other articles that contain the enzyme composition of the present invention. The present invention also relates to methods of preparing the enzyme compositions of the present invention.

It is to be understood that both the foregoing general description and the following detailed description are exemplary and explanatory only and are intended to provide a further explanation of the present invention, as claimed.

### DETAILED DESCRIPTION OF THE PRESENT INVENTION

The present invention is an enzyme composition that has a protective element for protection of an enzyme from denaturation. More specifically, the enzyme composition includes polymers functioning at least in part as a protective element, to an enzyme in such a way that the enzyme is heat stable, but is also capable of being released from the polymer, during certain conditions, so as to be able to engage in catalytic activities. In one embodiment, the present invention relates to a solid enzyme composition which contains at least one enzyme and at least one polymer. The enzyme, when present in the solid enzyme composition, is preferably stable at temperatures of at least 100° C. or higher. More preferably, the enzyme is stable at temperatures of at least 110° C. and even more preferably at least 120° C. when present in the solid enzyme composition. Most preferably, the enzyme is protected against degradation at temperatures of from about 100° C. to about 150° C. or to about 200° C. A heat stable enzyme can be an enzyme encased in polyamide that is a solid and that retains its non-denatured catalytic activity at a given temperature for a given period of time.

The polymer used in the present invention can be any suitable polymer. Preferably, the polymer of the present invention can be any polyamide polymer. This polyamide is preferably a solid at room temperature and more preferably has a melting point of at least 120° C., and more preferably, from 120° C. to 180° C. The polyamide polymer can be a polyamide copolymer. This polyamide polymer can be the product of the reaction between any suitable diacid with any suitable dibase. Preferably, the diacid can be any suitable C₄-C₁₂ diacid and more preferably a C₇-C₁₂ diacid and the dibase can be any suitable diamine, such as from 1,2-ethyl diamine to 1,12-dodecane diamine. Other examples include 1,7-diamino heptane, a 1,8 diamino octane, a 1,9-diamino nonane, a 1,10-diamino decane, a 1,11-diamino undecane, a 1,12-diamino dodecane or combinations thereof. (In the designation of diamines herein, both the listing of the alkyl first and the diamine second, and the listing of the diamine first and the alkyl second are used interchangeably.) Non-limiting examples of combinations of diacids and diamines include the following: a diacid length of 6 to 12 carbons with a diamine length of 3 to 6 carbons; a diacid length of 8 to 12 carbons with a diamine length of 4 to 6 carbons, and most preferably, a diacid length of 8 to 12 carbons with a diamine length of 3 to 4 carbons. Table 1 details specific examples of suitable combinations of acids and bases capable of being used to produce polymers used in the present invention. In this listing, the diacid/diamine combinations that provide a polymer composition with the best steam resistance are azelaic acid/1,3-diaminopropane and sebacic acid/1,3-diaminopropane.

TABLE 1
Diacid and Diamine combinations for polyamide preparation

| | | Amount | | | Amount | |
|---|---|---|---|---|---|---|
| Acid | F.W. (g/mol) | Acid (gm) | Base | F.W. (g/mol) | Base (gm) | Ratio |
| Glutaric | 132 | 150 | 1,3-diaminopropane | 74 | 74 | 1.14:1 |
| | | 152 | 1,4-diaminobutane | 88 | 88 | 1.15:1 |
| | | 158 | 1,6-diaminohexane | 116 | 116 | 1.2:1 |
| Adipic | 146 | 166 | 1,3-diaminopropane | 74 | 74 | 1.14:1 |
| | | 168 | 1,6-diaminohexane | 116 | 116 | 1.15:1 |
| | | 175 | 1,8-diaminooctane | 144 | 144 | 1.2:1 |
| Pimelic | 160 | 182 | 1,4-diaminobutane | 88 | 88 | 1.14:1 |
| Azelaic | 188 | 214 | 1,3-diaminopropane | 74 | 74 | 1.14:1 |
| | | 216 | 1,4-diaminobutane | 88 | 88 | 1.15:1 |
| Sebacic | 202 | 230 | 1,3-diaminopropane | 74 | 74 | 1.14:1 |
| | | 232 | 1,4-diaminobutane | 88 | 88 | 1.15:1 |

Preferably, the polymers of the present invention can have a molecular weight from about 5,000 Daltons to about 500,000 Daltons, such as 10,000 Daltons to 50,000 Daltons or more and the enzymes of the present invention can have a molecular weight of from about 10,000 Daltons (or less) to about 15,000 Daltons or more. Preferably, the polymers are rigid (e.g., at ambient temperatures) and preferably do not exhibit traditional thermoplastic, pliable, or malleable properties. Also, preferably no rheological agents are used in the polymer matrix.

An important aspect of the present invention is that the polymer used should not denature the enzyme it is intended to protect. Furthermore, it is also preferable, but not required, that the polymer be non-toxic in nature. Finally, the polymer is preferably capable of releasing the protected enzyme once the catalytic function of the enzyme is desired.

Any suitable enzyme can be used in the present invention. More than one enzyme can be present. The enzyme(s) is preferably steam stable. The enzyme can be a dried particulate enzyme. For example, proteases, neutral proteases, alkaline proteases, acid proteases, esterase, mannosidase, glucosidase, galactosidase, amindase, transamindase, phosphatase, fucosidase, phytase, glucosaminidase, xylanases, amylases, pectinases, isomerases, oxidases, glucanases, cellulases, lipases, and the like may be used. The enzymes may be water soluble, water dispersible, water emulsifiable, water extractable or water insoluble. The enzymes may also be in any suitable non-fluid state including, powdered, prilled, granulated, microencapsulated, microcrystalline, membrane bound, particulate absorbed or particulate grafted and the like. The enzymes of the present invention may also have any suitable molecular weight. In the present invention, the enzyme is preferably protected from degradation at temperatures above 125° C.

In the present invention, the polymer can protect an enzyme by any suitable method. Methods of protection may include encapsulation, micro-encapsulation, entrapment, and the like. The polymer portion of the enzyme/polymer composition (enzyme composition) can make up from about 0.1% to about 99% by wt. of the total weight of the enzyme composition, such as at least about 50% by weight based on the total weight of the enzyme composition. Preferably, the polymer can make up from about 25% to about 75% by wt. of the enzyme composition. More preferably, the polymer can make up from about 40% to about 60% by wt. of the enzyme composition. One further aspect of the polymer of the present invention is that the polymer can release the enzyme in its active state when needed. For example, the polymer, when exposed to water (preferably, copious amounts of water) breaks down and/or dissolves and thereby releases the enzymes in the enzyme composition. As stated, the enzyme is releasable from the enzyme composition, preferably, to perform catalytic activities for any of a variety of purposes. Generally, the enzyme is releasable upon re-hydrating of the enzyme composition. For instance, the enzyme can be released within 30 minutes upon re-hydration.

The enzyme composition may take any suitable form. More specifically, the enzyme composition is a solid composition, such as a solid filament, solid flake, solid granule, solid nugget, solid bead, solid bar, and the like, or combination thereof. The enzyme composition can be an extruded product.

The enzyme component of the enzyme composition may be distributed in the enzyme complex in any suitable manner. For instance, the enzyme can be uniformly distributed throughout the polymer portion of the enzyme composition.

As stated, the enzyme(s) is protectable or protected during a sterilization process, for instance. Preferably, the enzyme exhibits at least 90% of the activity after 5 minutes once the enzyme composition containing the enzyme is exposed to a steam treatment. More preferably, the enzyme exhibits at least 95% and even more preferably from about 95% to about 99% of its activity in the enzyme composition after being exposed to a steam treatment.

The enzyme composition can be made by any suitable method. The following is an example of one possible method of making the enzyme composition and is intended for illustrative purposes only, and is not intended to limit the scope of the present invention.

Generally, the first step in the method of the present invention can be the combination of at least one acid and at least one base. The diacid can be mixed with the diamine in any fashion. In general, in making the enzyme compositions of the present invention, one can mix at least one C₇-C₁₂ diacid or other diacid with at least one diamine. Then, the temperature of the diamine/diacid combination can be increased until the combination forms a polymer. Then, the polymer can be cooled and subsequently blended with one or more enzymes to form the enzyme composition. As previously mentioned, Table 1 lists several examples of diacids and diamines that are particularly suitable for use in the present invention. Table 1 also lists the respective amounts of each chemical that are also particularly suitable for the present invention. The present invention is not limited to any of the specifics listed therein, as these examples are listed for illustrative purposes only.

The combination of the diacid and diamine, in a suitable reaction vessel can be accomplished by any suitable method. The diamine can be in a liquid form and can be heated to any suitable temperature. A suitable temperature is from about 50° C. to about 120° C. Most preferably, suitable temperature ranges are from about 70° C. to about 100° C. Other temperatures can be used. A suitable diacid, which can be a solid diacid, can then be added to the diamine.

Following the combination of the diacid and diamine, the temperature of the resulting mixture can be adjusted to any temperature that melts the diacid, if not already melted or in a liquid form. Preferably, the temperature is from about 120° C. to about 160° C. More preferably, this temperature is from about 130° C. to about 150° C. Generally, at this point, the diamine/diacid combination forms one or more salt acid/base complexes. At this point, the temperature can be maintained or can be adjusted to a temperature of from about 175° C. to about 210° C. until the diamine/diacid combination forms a polymer. The polymer can then be maintained at a temperature of from about 180° C. to about 190° C. or any other suitable temperature. This temperature can be applied for any suitable amount of time. A suitable amount of time can be any amount of time that preferably allows for the formation of the various salt complexes resulting from the various acid/base reactions and the like.

As an option, the next step in the reaction can be a further adjustment of the temperature of the above-mentioned diacid/diamine mixture to any temperature, for any suitable time period, which would facilitate melt polycondensation, and thereby the formation of the desired polymer. For example, the temperature can be increased to from about 165° C. to about 220° C. More preferably, the temperature can be increased to from about 175° C. to about 210° C., for instance 185° C. This temperature can typically be maintained for from about 0.5 to about 3 hours. More preferably this temperature is maintained for about 1 to about 2.5 hours. Other temperatures and/or times can be used.

The composition described in the above-mentioned paragraph can be further treated to remove any processing water present therein by any suitable method. More preferably, this processing method can be a vacuum water removal method utilizing sufficient suction for a sufficient length of time, to remove any processing water present in the polymer composition. This process and all of the above-mentioned processes can be performed, and preferably are performed while the composition is under agitation.

Once the diacid and diamine components have polymerized, thereby forming a polymer composition, the temperature of the polymer composition can be reduced. This temperature reduction can be done at any suitable rate, but is more preferably done slowly over several stages. The purpose of staged cooling, besides the protection of certain enzymes that will be added, is to allow the temperature to be reduced uniformly throughout the polymer composition. As an example, the polymer composition can be cooled from a temperature of about 210° C. to a temperature of from about 140° C. to about 160° C., such as to about 150° C. The polymer composition can be maintained at this temperature until its centipoises (Cp) value is from about 20,000 to about 40,000. As another example, the polymer composition can be maintained at 130° C. until the Cp value is from about 25,000 to about 35,000.

The temperature of the polymer composition can then be further adjusted to from about 140° C. to about 120° C., such as to about 130° C. The polymer composition can be maintained at this temperature until its centipoises (Cp) value is from about 40,000 to about 110,000. For example, the polymer composition can be maintained at a temperature of from about 125° C. to about 135° C. until the Cp value is from about 50,000 to about 100,000.

The polymer composition can then be gradually adjusted to from about 70° C. to about 140° C., such as to about 80° C. to about 110° C. This temperature range can be adjusted by one skilled in the art depending on the temperature tolerances of the specific enzyme used and the temperature range for polymer plasticity for the particular polymer used.

After the preparation and cooling of the polymer composition, the enzyme can be added to the polymer by any suitable method. Once added, the enzyme can be blended with the polymer by any suitable method. More specifically, the enzyme can be blended into the polymer while the polymer is in a melted state, until a homogeneous polymeric enzyme composition is formed. As a specific example, the enzyme can be added while the polymer composition is maintained at a temperature of about 120° C.-125° C. and can be distributed throughout the polymer by agitation to form a homogeneous composition. The temperatures used allow the enzyme to be distributed by, for instance, agitation throughout the polymer, to preferably achieve homogeneity, and this also permits the composition to be poured or extruded for molding purposes.

Once the enzyme composition has been formed, it can be shaped into any suitable form. For example, the enzyme composition can be run through a cooling extruder and formed into solid filaments, solid flakes, solid granules, solid beads, solid bars, or any other extruded shape or combinations thereof. Typically, the enzyme composition will become solid at a temperature of from about 90° C. to about 120° C. and will be a solid at room temperature.

The enzyme composition may be used in any number of suitable applications, depending on the selection of the enzyme. For example, the enzyme composition can be added to human or pet food products to assist in the digestion of the same. In particular, the present invention can be used in dog and cat food. For instance, the enzyme composition of the present invention can be present in an amount of from about 100 mg to about 1000 mg per pound of dog food. Upon digestion, the polymeric composition present in the enzyme composition breaks down thereby releasing the enzyme for digestion. Thus, the present invention provides a beneficial way to protect enzymes during the incorporation of the enzymes in various food products and other non-food products and the enzyme is protected during heat treatments or other types of sanitary processing and yet the enzyme can then be released upon contact with water or other aqueous solutions. An example of an enzyme used in food production is given, for example, in U.S. Pat. No. 4,804,549. The present invention can be used in cooling water applications where enzymes, for example, may be fashioned into stabilized pellets, granulated polymer coated particles, tablest, pucks, or other shape forms. In all these instances, the enzyme is encased in solid polymer and the polymer with encased enzyme may be fashioned into any form designated. The form selected facilitates ease of handling, no dusting properties, no splash and spill concerns, and may be premeasured sizes for appropriate delivery concentrations to a volume of process cooling water.

In agriculture, for example, the solid stabilized enzyme material may be granular, particulate, tableted, or other forms which can be delivered to an aqueous make down tank for dilution purposes wherein the solid encased enzyme is released from the solid polymer upon immersion into water. Upon immersion, the enzyme is released in its active state.

In leather processing, the solid encased enzyme is a granular, pelleted, or extruded solid form may be added to processing drums with animal hides in an aqueous solution. The enzyme is released from its polymer when immersed in water and then it becomes active and free to degrease, unhair, or condition the skin of the animal hide during the process period.

In animal feed processes, the feed is required to undergo steam disinfection for several minutes. Enzymes are added to animal feed for predigestion of the feed once ingested by the animal. The steam process destroys most enzymes as it disinfects the food stuffs. By encasing the enzymes in a solid polymer resistant to steam, the active integrity of the enzyme is maintained for predigestion of the food stuffs.

The present invention can also be used, for example, in cooling process waters, as, for example, a biocide, in agricultural development, as, for example, a fertilizer composition, and in leather processing and the like. A wide range of industrial uses for enzymes is disclosed in, for example, U.S. Pat. No. 6,342,381, which particularly discusses industrial uses of proteases, xylanases, amylases, pectinases, isomerases, oxidases, beta-glucanases, cellulases, hemicellulases, lipases, phospholipases, redox enzymes and the like. The compositions of U.S. Pat. No. 6,342,381 are in liquid form. The following are specific examples of industrial uses of enzymes: as biocides, for example, to eliminate bacteria, algae and fungi, from bodies of water; as a treatment for waste products to convert them into useful organic materials such as sugars, lignan, ethanol and thermoplastics; as compositions for fiber degradation, such as, for example in papermaking processes; as esterase compositions to hydrolyze fats and oils, such as, for example, lipases used in reducing pitch deposits on rolls and other equipment in a papermaking process and as a degreaser in a leather making process. One can readily appreciate instances wherein it would be preferable to have an enzyme preparation in solid form as provided by the present invention, because a solid form is required by a particular use of the enzyme composition, or because a solid form is required for storage or transporting the enzyme composition. Further, one can readily appreciate instances wherein it would be preferable to have an enzyme composition that protects an enzyme from temperatures of 100° C.-200° C., and particularly that protects an enzyme from steam conditions encountered in some industrial processes. All of the patents/publications mentioned herein are incorporated herein by reference.

The present invention will be further clarified by the following examples, which are intended to be exemplary of the present invention.

### EXAMPLES

Diacid and diamine combinations for polyamide copolymers listed in Table 1 are characteristic of the type of preparations which perform well as stabilizers for steam exposed enzymes. The amount of diacid and diamine used was based on a weight ratio of 3.125 diacid to 1 diamine. Other ratios can be used such as a diacid ratio of 3.125 to 2.630 to a dibase of 1 (one). Among this inclusive listing, the diacid/diamine combinations which provide the best steam stability for enzymes are azelaic acid/1,3-diaminopropane and sebacic acid/1,3-diaminopropane and pimelic acid/1,4-diaminobutane. The following description details the production of sebacic acid/1,3-diaminopropane copolymer and this production protocol is typical of all diamine/diacid combinations listed in Table 1 with minor adjustments in ratio. The diamine can be in liquid form or if originally in solid form. Any solid can be heated to a melt temperature to produce the liquid form. In the present example, 1,3-diaminopropane was heated to a temperature of 100° C. to approximately 120° C. and the sebacic diacid solid was then added to the diamine. Upon combining the diacid and diamine, the temperature of the resulting mixture was adjusted to a temperature that melted the solid diacid. In this example, the sebacic acid was heated to a temperature of 130° C. to approximately 150° C. At the melt temperature of the diacid, the diacid/diamine combination forms one or more salt acid/base complexes. A blanket of nitrogen was maintained above the acid/base complex and the temperature was adjusted to 175° C. or higher until steady condensation between salt complexes began to polymerize. Steady polymerization was indicated by the production of water which evolved from the condensation reactions. The water was released as vapor and bubbled to the top of the reaction vessel. At this time the nitrogen blanket was discontinued and a slight vacuum was pulled to relieve the evolved water vapor from pressurizing in the vessel. The temperature was slowly raised until about 210° C. was reached. The reaction was then lowered to between 180° C. and 190° C. and maintained for 0.5 to 3 hours until the water vaporizing activity had subsided. When most of the water vapor was removed via vacuum; the reaction temperature was lowered in stages to allow uniform cooling of the polymer composition throughout the reaction vessel. The polymer composition was cooled to between 150° C. and 160° C. where it was maintained until its centipoise value was between 25,000 to 35,000. Over the following hour the temperature was slightly lowered to about 135° C. and the centipoise value was between 50,000 and 100,000.

After this preparation and cooling the neutral protease (Neutrase) and/or the alkaline protease were added to the polymer composition by any suitable method. The enzymes were blended into the polymer composition while the polymer was in the liquid state and were distributed throughout the polymer by agitation to achieve homogeneity. The enzymes were originally in a granular state and this granular state was maintained inside the polymer composition. Once the enzyme/polymer composition had been formed the composition was shaped into flaked chips by means of extrusion molding. The extrusion molded enzyme/polymer complex cooled instantly upon flake chipping and was now in the final form for steam resistance treatment.

Examples of neutral protease and alkaline protease enzymes prepared by the previously described method were tested for their enzyme protecting capacity in a steady flow of steam for an hour; where the enzyme activity was monitored before and after steam treatment. The results of these treatments were observed and the results provided below.

As an example of thermal stability which is confered upon a protein incorporated into the polymer matrix, proteolytic activity assessment was performed via use of a Hide Powder Azure (HPA) substrate. A 0.1M phosphate buffer solution (Sorenson) pH 7.8-8.0 and amended with approximately 200 milligrams Triton—X-100 surfactant served as the incubation solution for the (HPA) substrate. The incubation temperature was 40° C.+/−2° C. for 20 minutes. HPA was introduced into the incubation mix at approximately 20 mg/2.5 ml incubation solution. At the end of incubation, the dispersion was filtered through a 0.45 micron filter to remove particulates and effectively stop the reaction. The filtrate was read for absorbance at 595 nanometers (nm) in a 1 cm cell path spectrophotometer. “HPA activity unit-U hpa” was defined as the amount of enzyme that under test conditions, caused a change of 0.1 Abs 5959 unit/minute.

Before and after thermal testing of polymer protein matrix complex. Thermal challenge at approximately 100° C.+/−2 C for 60 minutes.

| | | | Specific Activity | |
|---|---|---|---|---|
| | Total protein | Protein concn. | mg HPA/min/mg | Activity/gm |
| Product | Mg/g product | in assay as ug/ml | Protein | product |
| Polymer/Neutrase 0.8 L | 70.0 | 3.24 | 436.72 | 30,570 |
| (Before) | | | 459.87 | 32,191 |
| | | | 451.54 | 31,607 |
| | | | 440.80 | 30,856 |
| Polymer/Neutrase 0.8 L | 70 | 3.24 | 438.61 | 30,702 |
| (After) | | | 419.38 | 29,357 |
| | | | 426.43 | 29,850 |
| | | | 468.77 | 32,814 |
| Polymer/Neutrase MG | 95.9 | 3.24 | 648.14 | 62,157 |
| (Before) | | | 598.28 | 57,375 |
| | | | 609.62 | 58,462 |
| | | | 631.17 | 60,529 |
| Polymer/Neutrase MG | 95.9 | 3.24 | 660.73 | 63,364 |
| (After) | | | 628.89 | 60,310 |
| | | | 611.23 | 58,616 |
| | | | 593.57 | 56,923 |

Therefore, no significant loss of activity was detected in the (After) treatments.

In a second example, an alkaline protease was incorporated into the polymer matrix and proteolytic activity was recorded (Before and After) treatment.

Before and after thermal testing of alkaline protease polymer matrix. Thermal challenge at approximately 100° C.+/−2° C. for 60 minutes.

| | | | Specific Activity | |
|---|---|---|---|---|
| | Total protein | Protein concn. | mg HPA/min/mg | Activity/gm |
| Product | Mg/g product | in assay as ug/ml | Protein | product |
| Polymer/Alk. Protease | 88.3 | 3.10 | 481.62 | 42,527 |
| Before | | | 506.23 | 44,700 |
| | | | 473.81 | 41,837 |
| | | | 489.74 | 43,244 |
| | | | 494.68 | 43,680 |
| Polymer/Alk. Protease | 88.3 | 3.10 | 474.93 | 41,936 |
| After | | | 483.12 | 42,659 |
| | | | 496.37 | 43,829 |
| | | | 498.56 | 44,023 |
| | | | 480.78 | 42,453 |

Again there was no significant difference in the before and after treatment of the polymer/Alk. Protease matrix complex.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

What is claimed is:

1. A solid enzyme composition comprising at least one enzyme and at least one polymer, wherein said at least one enzyme is stable at a temperature of at least 100° C.

2. The enzyme composition of claim 1, wherein said polymer is a polyamide polymer having a melting point of 120° C. or higher.

3. The enzyme composition of claim 2, wherein said at least polyamide polymer is a polyamide copolymer.

4. The enzyme composition of claim 3, wherein said at least polyamide polymer is formed from reacting at least one diacid with at least one diamine.

5. The enzyme composition of claim 4, wherein said diacid is a C₇-C₁₂ diacid and said diamine is a 1,2 ethyl diamine to a 1,12 dodecane diamine.

6. The enzyme composition of claim 4, wherein said diacid is a glutaric acid, adipic acid, pimelic acid, azelaic acid, sebacic acid, or combinations thereof.

7. The enzyme composition of claim 4, wherein said diamine is a 1,7-diamino heptane, a 1,8 diamino octane, a 1,9-diamino nonane, a 1,10-diamino decane, a 1,11-diamino undecane, a 1,12-diamino dodecane or combinations thereof.

8. The enzyme composition of claim 2, wherein said at least polyamide polymer has a molecular weight of from about 5,000 to about 20,000 daltons.

9. The enzyme composition of claim 2, wherein at least one polyamide polymer has a molecular weight of from about 10,000 to about 15,000 daltons.

10. The enzyme composition of claim 2, wherein said at least one polyamide is present in an amount of from about 0.1% to about 99% by weight based on the total weight of said enzyme composition.

11. The enzyme composition of claim 2, wherein said at least one polyamide polymer is present in amount of from about 25% to about 75% by weight based on the total weight of the enzyme composition.

12. The enzyme composition of claim 2, wherein said at least polyamide polymer is present in an amount of at least about 50% by weight based on the total weight of said enzyme composition.

13. The enzyme composition of claim 1, wherein said enzyme composition has a shape that is a filament, flake, granule, nugget, bead, bar, or combinations thereof.

14. The enzyme composition of claim 1, wherein said enzyme composition is an extruded product.

15. The enzyme composition of claim 2, wherein said at least one enzyme is uniformly distributed throughout said at least polyamide polymer.

16. The enzyme composition of claim 1, wherein said at least one enzyme is steam stable.

17. The enzyme composition of claim 1, wherein said at least one enzyme is protected from degradation at temperatures above 125° C.

18. The enzyme composition of claim 1, wherein said at least one enzyme is protected against degradation at temperatures of from about 100° C. to about 150° C.

19. The enzyme composition of claim 1, wherein said at least one enzyme is releasable from said enzyme composition.

20. The enzyme composition of claim 1, wherein said at least one enzyme is releasable from said enzyme composition to perform catalytic activities.

21. The enzyme composition of claim 1, wherein said at least one enzyme is releasable upon re-hydrating of said enzyme composition.

22. The enzyme composition of claim 1, wherein said at least one enzyme is released within 30 minutes upon re-hydration.

23. The enzyme composition of claim 1, wherein said enzyme is a protease, an esterase, a xylanase, an amylase, a pectinase, an isomerase, an oxidase, a beta-glucanase, a cellulase, a hemicellulase, a lipase, a phospholipase, a redox enzyme, a mannosidase, a glucosidase, a galactosidase, an amindase, a transamindase, a phosphatase, a fucosidase, a phytase, a glucosaminidase, or combinations thereof.

24. A food composition comprising at least one enzyme composition of claim 1 and at least one foodstuff.

25. The food composition of claim 23, wherein said foodstuff is animal food.

26. The food composition of claim 23, wherein said foodstuff is dog food.

27. A biocide composition wherein the biocide comprises the enzyme composition of claim 1.

28. A composition for treating a waste product to obtain useful organic products therefrom, wherein the composition comprises the enzyme composition of claim 1.

29. A degreasing composition for treating hides and pelts in a leather-making process wherein the composition comprises the enzyme composition of claim 1 and wherein the enzyme is a lipase.

30. A composition for hydrolyzing fibers in a papermaking process, wherein the composition comprises the enzyme composition of claim 1 and wherein the enzyme is a cellulase.

31. The enzyme composition of claim 1, wherein said at least one enzyme is a dried particulate enzyme.

32. The enzyme composition of claim 1, wherein at least one enzyme is protectable or protected during a sterilization process.

33. The enzyme composition of claim 1, wherein said at least one enzyme is water-soluble, water-dispersible, water-emulsifiable, water-extractable, or water-insoluble.

34. The enzyme composition of claim 1, wherein said enzyme is a powder, prilled, granulated, micro-encapsulated, micro-crystalline, membrane bound, particulate adsorbed, or particulate grafted enzymes, or combinations thereof.

35. The enzyme composition of claim 1, wherein said at least one enzyme exhibits at least 90% activity after 5 minutes of said enzyme composition being exposed to a steam treatment.

36. A method for producing the enzyme composition of claim 1, comprising: a) mixing at least one C₇-C₁₂ diacid and at least one diamine; b) increasing the temperature of said diamine/diacid combination until said diamine/diacid combination forms a polymer that is a solid when cooled to room temperature; c) cooling said polymer to an intermediate temperature above room temperature; and d) blending an enzyme with said polymer thereby forming said enzyme composition.

37. The method of claim 36, wherein the diamine of step a) is at a temperature of from about 70° C. to about 100° C.

38. The method of claim 36, wherein the temperature of step b) is from about 130° C. to about 150° C. until said diamine/diacid combination forms one or more salt acid/base complexes, then said temperature is maintained at from about 175° C. to about 210° C. until said diamine/diacid combination forms a polymer, then the polymer is maintained at a temperature from about 180° C. to about 190° C.

39. The method of claim 38, further comprising applying a vacuum to said polymer while said temperature is being maintained at from about 180° C. to about 190° C., said vacuum being of sufficient strength and being applied for a sufficient time to remove any processing water from said polymer.

40. The method of claim 36, wherein said cooling of said polymer occurs in stages; a first stage wherein the temperature of said polymer is maintained at from about 140° C. to about 160° C. until the centipose value of said polymer is from about 20,000 to about 40,000; followed by a second stage where the temperature of said polymer is maintained at from about 125° C. to about 135° C. and maintained at that temperature until said polymer has a centipose value of from about 50,000 to about 100,000; followed by a fourth stage wherein the temperature of said polymer is maintained at from about 70° C. to about 140° C.

41. The method of claim 36, further comprising forming said enzyme composition into one or more shapes.

42. The method of claim 36, wherein said diamine in step a) is a liquid and said diacid in step b) is a solid.

43. The method of claim 36, wherein said polymer is polyamide.

44. The method of claim 36, wherein said diacid is glutaric, adipic, pimelic, azelaic, sebacic or any combination thereof.

45. The method of claim 36, wherein said diamine comprises 1,3-diaminopropane; 1,4-diaminobutane; 1,6-diaminohexane; 1,8-diaminooctane or a combination thereof.

46. The method of claim 36, wherein the diacid is azelaic acid or sebacic acid and the diamine is 1,3-diaminopropane.
